(19) United States
(12) Patent Application Publication
(10) Pub. No.: US 2001/0028897 A1
(43) Pub. Date: Oct. 11, 2001

(54) Compositions and treatment methods for benign prostatic hypertrophy

(76) Inventor: Milton Hammerly, Parker, CO (US)

Correspondence Address: Christopher J. Whewell, Western Patent Group, 6020 Tonkowa Trail, Georgetown, TX, 78628, US

(21) Appl. No.: 09/784,584

(22) Filed: Feb. 15, 2001

Related U.S. Application Data
(60) Provisional application No. 60/183,703, filed on Feb. 18, 2000.

(51) Int. Cl. A61K 35/78; A61K 31/517

(52) U.S. Cl. 424/727; 514/259; 514/252.17

(57) ABSTRACT

Provided herein are medicinal compositions of matter that comprise an alpha-1 adrenergic blocker in combination with an extract derived from the berries of the Saw Palmetto plant, and methods for their administration. The extract is typically obtained from the dried berries using extraction means known to those skilled in the art. Through use of the compositions of the invention, relaxation of the smooth muscles present in the prostate gland may be effected while simultaneously inhibiting glandular growth of the prostate thus providing a heretofore unobserved synergy in terms of relief to sufferers of benign prostatic hypertrophy.

This application claims the benefit of U.S. Provisional Application Ser. No. 60/183,703 filed Feb. 18, 2000 the entire contents of which are herein incorporated by reference.

### TECHNICAL FIELD

This invention relates to a medicinal composition of matter comprising any one of known alpha-1 adrenergic blocking drugs in combination with Saw Palmetto. The compositions according to the invention are suitable for oral administration. Alternatively, the combinations of the invention may be administered by other means including without limitation intravenously, intramuscularly, subcutaneously, sublingually, buccally, and parenterally.

### BACKGROUND

Benign prostatic hypertrophy (“BPH”) is a medical condition that affects nearly all men as they age. BHP is a condition characterized by a nodular enlargement of prostatic tissue resulting in obstruction of the urethra. The incidence of BPH is clearly age related. Based on an examination of autopsy specimens, a 30 percent incidence of benign hyperplasia was noted in the prostates of men aged 60-69, and a 100 percent incidence from men overa 90 (Randall and Hinman, 1983). This correlates with a 35 percent incidence of palpable prostatic enlargement during physical examination of men aged 60-69 Lytton, 1983). It is estimated that half of all men over 65 have some prostatic enlargement, and at least one third of these will have clinical symptoms of BPH. In vies of the continually increasing life span, the number of BPH patients and therefore the impact of this disease will continue to increase. Although BPH is currently treated primarily via surgical techniques, there is both clinical and experimental evidence to suggest that pharmacological management of this disease is possible and can delay or prevent the necessity of surgery in a significant percentage of patients. BPH is commonly associated with increased urinary frequency, decreased urine flow, and dribbling. Additionally, the condition frequently requires aging men to awaken on several occasions during the night to urinate. With respect to the pharmacological treatments available, the medical community currently recognizes one of two possible strategies for treating the symptoms of BPH.

One approach at non-surgical treatment of BPH involves the administration of compounds known as alpha-1 adrenergic blockers. These materials improve urine flow by causing relaxation of the smooth muscles present in the prostate gland. Exemplary compounds of this class comprise, without limitation: Cardura (doxazosin), Flomax (tamsulosin), and Hytrin (terazosin).

Another approach involves the administration of compounds which inhibit the intracellular enzyme responsible for conversion of androgens (such as testosterone) in the prostate. In the case of testosterone, the enzyme 5-alpha reductase causes the testosterone to be converted to 5-alpha-di-hydroxytestosterone (“DHT”). One such compound capable of inhibiting this enzyme is known as Proscar (finasteride). By interfering with such androgen conversion, the growth of the glandular component of BPH is inhibited. This inhibition can hinder or stop the natural progression of BPH and may even cause some regression of prostatic hypertrophy, which in turn results in improved urine flow and fewer trips to the bathroom at night. However, the relatively high cost of Proscar, combined with its undesirable side effects and slower onset of action (as compared to the alpha-1 adrenergic blockers) has made it a less popular choice.

U.S. Pat. No. 4,330,559 teaches a method for treating BPH which comprises the administration of 2,5-di-amino-2-(mono-, di-, or trifluoromethyl) pentanoic acids or derivatives thereof. A preferred method includes the administration of 2,5-diamino-2-difluoromethylpentanoic acid. Thus, it is seen that certain amino carboxylic acids are useful in the treatment of BPH.

U.S. Pat. No. 4,342,750 discloses a process for the treatment of benign prostatic hypertrophy in a large mammal afflicted with benign prostatic hypertrophy which comprises orally administering to the mammal an effective dose for treating benign prostatic hypertrophy of n-acyl amphotericin B.

U.S. Pat. No. 4,237,117 sets forth a method for the treatment of benign prostatic hypertrophy in human males, comprising administering an active compound selected from the group consisting of partricin, C₁-C₆ alkyl ester of partricin and C₁-C₆ alkyl ester of N-mono and N,N-disubstituted partricin, the N-substituents being C₁-C₆ alkyl or carboxylic acyl, to a human male in need of such treatment in an amount sufficient to alleviate benign prostatic hypertrophy.

U.S. Pat. No. 4,055,641 discloses the use of steroidal compounds described by the formula:

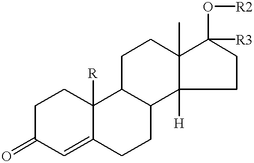

wherein R is —CHO or —CH₂OR₁; each of R₁ and R₂ is hydrogen, alkylcarbonyl wherein the alkyl moiety has from 1 to 20 carbon atoms and is straight or branched, benzoyl, phenylalkylcarbonyl wherein the alkyl moiety has from 1 to 6 carbon atoms and is straight or branched or cycloalkylcarbonyl wherein the cycloalkyl moiety has from 5 to 10 carbon atoms; R₃ is hydrogen; or R₂ and R₃ together form a double bond between the 17- position carbon atom and the oxygen atom.

U.S. Pat. No. 4,039,661 teaches a process for treating benign prostatic hypertrophy in a mammal afflicted with benign prostatic hypertrophy which comprises orally administering to said mammal an effective amount for treating benign prostatic hypertrophy of antibiotic SCH-16656.

The foregoing U.S. patents are herein incorporated in their entirety by reference thereto.

It is well known that benign prostatic hypertrophy is marked by an abnormal elevation of polyamine levels (see for example, Cancer Research, 38, 2321 (1978)). Hence, the polyamines are likely to play an important role in the causation or maintenance of benign prostatic hypertrophy. While not wishing to be bound by any particular theory, it is believed that by inhibiting the effects of the polyamines by blocking the formation of the polyamines or the receptor sites at which they interact, the interruption or arrest of the course of the enlargement of the prostate gland may be effected.

In benign prostatic hyperplasia, the male hormone 5.alpha.-dihydrotestosterone has been identified as possibly being the principal culprit. The continual production of 5.alpha.-dihydrotestosterone by the male testes induces incremental growth of the prostate glad throughout the life of the male. Beyond the age of about fifty years, in many men, this enlarged glad begins to obstruct the urethra with the pathologic symptoms noted above.

The elucidation of the mechanism summarized above has resulted in the recent development of effective agents to control, and in many cases reverse, the pernicious advance of BPH. In the forefront of these agents is Merck & Co., Inc.s' product Proscar® (finasteride). The effect of this compound is to inhibit the enzyme testosterone 5-alpha reductase, which converts testosterone into 5.alpha.-dihydrotesterone, resulting in a reduced rate of prostatic enlargement, and often reduction in prostatic mass.

The development of such agents as Proscar® bodes well for the long-term control of BPH. However, as may be appreciated from the lengthy development of the syndrome, its reversal also is not mediate. In the interim, those males suffering with BPH continue to suffer, and may in fact lose hope that the agents are working sufficiently rapidly.

In response to this problem, one solution is to identify pharmaceutically active compounds which complement slower-acting therapeutics by providing acute relief. Agents which induce relaxation of the urethral smooth muscle, by binding to alpha-1 adrenergic receptors, thus reducing the increased adrenergic tone due to the disease, would be good candidates for this activity. Thus, one such agent is alfuzosin, which is reported in EP 0 204597 to induce urination in cases of prostatic hyperplasia. Likewise, in WO 92/0073, the selective ability of the R(+) enantiomer of terazosin to bind to adrenergic receptors of the .alpha..sub.1 subtype was reported. In addition, in WO 92/161213, herein incorporated by reference, combinations of 5-alpha-reductase inhibitory compounds and alpha 1-adrenergic receptor blockers (terazosin, doxazosin, prazosin, bunazosin, indoramin, alfuzosin)were disclosed. However, no information as to the .alpha₁ₐ, alpha_{1b}, or .alpha_{1c} subtype specificity of these compounds was provided as this data and its relevancy to the treatment of BPH was not known. Current therapy for BPH uses existing non-selective alpha-1 antagonists such as prazosin (Minipress, Pfizer), Terazosin (Hytrin, Abbott) or doxazosin mesylate (Cardura, Pfizer). These non-selective antagonists suffer from side effects related to antagonism of the alpha-1a and alpha-1b receptors in the peripheral vasculature, e.g., orthostatic hypotension and syncope.

The combinations of this invention are used to reduce the acute symptoms of BPH. Thus, combinations of this invention may be used alone or in conjunction with a more long-term anti-BPH therapeutics, such as testosterone 5-alpha reductase inhibitors, including PROSCAR.RTM. (finasteride). Aside from their utility as anti-BPH agents, these compounds may be used to induce highly tissue-specific, localized .alpha..sub.1c adrenergic receptor blockade whenever this is desired. Effects of this blockade include reduction of intra-ocular pressure, control of cardiac arrhythmias, and possibly a host of alpha-1c receptor mediated central nervous system events.

### SUMMARY OF THE INVENTION

The invention provides medicinal compositions useful in the treatment of benign prostatic hypertrophy. The compositions according to the invention are formed from components comprising a first component comprising the extract of the berries of the Saw Palmetto plant and a second component comprising an alpha-1 adrenergic blocker. Such compositions may contain either component in a wide range of concentration levels, depending on the intended use. Thus, one aspect of the invention comprises a medicinal composition useful in the treatment of benign prostatic hypertrophy formed from components comprising: a) a first component comprising the extract of the berries of the Saw Palmetto plant; and b) a second component comprising an alpha-1 adrenergic blocker.

Another aspect of the present invention includes a process for treating a person afflicted with benign prostatic hypertrophy comprising simultaneous administration to such person a combination, which combination includes: a) a therapeutically effective amount of the extract of the berries of the Saw Palmetto plant for treating benign prostatic hypertrophy; and b) a therapeutically effective amount of an alpha-1 adrenergic blocker for treating benign prostatic hypertrophy. According to a preferred form of the invention, the alpha-1 adrenergic blocker may be selected from the group consisting of: terazosin, doxazosin, prazosin, bunazosin, indoramin, and alfuzosin. According to a preferred form of the invention, the extract contains between 0.20% and 0.40% sterols, including every hundredth percentage therebetween, and wherein said first component contains between 80.00% and 95.00% of fatty acids, including every hundredth percentage therebetween,

### DETAILED DESCRIPTION

The present invention provides combinations useful alone or in combination with other materials in the treatment of benign prostatic hypertrophy. Generally speaking, a combination according to the invention will include a first component that comprises the extract of the berries of the Saw Palmetto plant (Serenoa repens) and a second component which comprises an alpha-1 adrenergic blocker.

### Extract of Saw Palmetto

The Saw Palmetto plant (Serenoa repens) has been known for centuries as a grass-like plant which produces berries. Saw Palmetto berries typically contain about 8% to about 13% oil. Of the many extraction methods known to medicinal chemists, the most preferred means for extracting the oil from the Saw Palmetto are the “cold press” process and the “gas extraction” method, which are well known to those skilled in the art. Solvent extraction techniques are also useful, in which cases the most common solvents used are hexane, carbon dioxide, and ethanol. There are a variety of compounds within the Saw Palmetto berry (Fitzpatrick, 1995) which can be divided into four major categories, all of which are oil-soluble: 1) Free fatty acids, of which those present in highest concentration include oleic acid, lauric acid, myristic acid and palmitic acid; 2) Phytosterols (plant sterols), which are plant sterols that have a chemical structure similar to cholesterol. The most commonly found phytosterols in saw palmetto are beta-sitosterol, campesterol, stigmasterol and cycloartenol (Plosker, 1996); 3) Free fatty alcohols, which are typically made up of fatty acids joined to an alcohol molecule; and 4) Monoglycerides, which are single fatty acids attached to a three-carbon glycerol molecule (Shimada, 1997). The efficacy of Saw Palmetto in the treatment of BPH has been documented, and both the efficacy and the mechanism of action of Saw Palmetto are believed to be similar to those of Proscar, although the present invention should not be construed as being limited to this or any other theory. According to research, Saw Palmetto also has a slight antispasmodic action on smooth muscles and it has far fewer side effects than Proscar. Thus the advantages of Saw Palmetto over Proscar include its being readily available, without restrictions, to any manufacturer; its lower cost over Proscar; and its fewer side effects than Proscar.

A “typical dose” of Saw Palmetto, for purposes of this specification, useful for treating BPH is 160 milligrams twice daily of an extract of the dried berries of the Saw Palmetto plant, which extract has been standardized to contain between about 80.0% and 95.0% by weight of constituent fatty acids known by those skilled in the art to be present in Saw Palmetto, in addition to about 0.2% to about 0.4% of constituent sterols known by those skilled in the art to be present in Saw Palmetto.

### Alpha-1 Adrenergic Blockers

Human adrenergic receptors are integral membrane proteins which have been classified into two broad classes, the alpha and the beta adrenergic receptors. Both types mediate the action of the peripheral sympathetic nervous system upon binding of catecholamines, norepinephrine and epinephrine.

Norepinephrine is produced by adrenergic nerve endings, while epinephrine is produced by the adrenal medulla. The binding affinity of adrenergic receptors for these compounds forms one basis of the classification: alpha receptors bind norepinephrine more strongly than epinephrine and much more strongly than the synthetic compound isoproterenol. The binding affinity of these hormones is reversed for the beta receptors. In many tissues, the functional responses, such as smooth muscle contraction, induced by alpha receptor activation are opposed to responses induced by beta receptor binding. Thus, alpha-blockers work by relaxing the smooth muscle of the prostate and bladder neck to improve urine flow and reduce bladder outlet obstruction.

Although certain particular alpha-1 adrenergic blockers have been described as representative examples of materials within the broad class of alpha-1 adrenergic blockers, their mention should not be construed as limiting the invention in any way. In fact, the invention comprises the use of any alpha-i adrenergic blockers in combination with Saw Palmetto as herein described. Thus, the term “alpha-1 adrenergic blocker” as used in this specification and the claims appended hereto means any alpha-1 adrenergic blocker that is recognized by those skilled in the art to have therapeutic utility as an alpha-1 adrenergic blocker, including without limitation terazosin, doxazosin, prazosin, bunazosin, indoramin, and alfuzosin.

### Combinations According to the Invention

The present invention is concerned with a physical mixture of an extract of Saw Palmetto and an alpha-1 adrenergic blocker. A mixture according to the invention may be employed by itself as an intermediate to which other therapeutically active materials may be added, or they may be used alone as therapeutic agents. Other therapeutically active materials may include a second alpha-i blocker, vitamins, colorants, binding agents, etc. Alternatively, an elixir may be made containing Saw Palmetto extract, an alpha-1 blocker, and a solvent in which both are soluble, such as ethanol, and adjusted to any desired concentration of the constituents.

To make a composition according to the invention, one selects the desired amounts of each component and physically mixes the two components, using mixing means known to those skilled in the art, which may include mechanical mixing. In one preferred embodiment, the mixing is conducted at a temperature in the range of about 25 degrees centigrade to 40 degrees centigrade, with about 40 degrees centigrade being most preferred during the mixing process, and the mixture is cocomminutated until a uniform mixture results. By combining an alpha-1 blocker with Saw Palmetto oil, a new product composition is provided which affects both the muscular and the glandular components of BPH to a patient to whom it is administered. The result is a clinically safe product that is clinically more effective than compositions of the prior art used to treat BPH, well tolerated by nearly all patients, and very reasonably priced.

According to the invention, the “typical dose” or any other dose amount of Saw Palmetto extract may be combined with a therapeutically effective amount of alpha-1 adrenergic blocker to create a dual-acting synergistic product that treats both the muscular and glandular elements of BPH. Thus, a composition according to the invention may comprise any and all dosage amounts of Saw Palmetto recognized by those skilled in the art as being therapeutically effective in combination with any and all dosage amounts of alpha-1 adrenergic blockers recognized by those skilled in the art as being therapeutically effective.

According to a preferred form of the invention, the amount of alpha-1 adrenergic blocker present in a composition that is to be administered to a patient is the same as normally prescribed for the particular alpha-1 adrenergic blocker selected. According to a preferred form of the invention, the amount of Saw Palmetto extract present in a combination according to the invention that is to be administered to a patient is about 160 milligrams per dose, when the Saw Palmetto extract contains between 80.0% and 95.0% of fatty acids and between 0.20% to 0.40% of sterols.

### EXAMPLE 1

16.0 grams of Saw Palmetto Extract containing between 80.0% and 95.0% of fatty acids and between 0.20% to 0.40% of sterols is dissolved in 100 milliliters of 90% ethanol in a 250 ml beaker. To this solution is added 0.12 grams of alfuzosin and admixture is effected by gentle stirring of the solution. Removal of the excess solvent provides a residue suitable for the treatment of benign prostatic hypertrophy. A typical dose for an adult human is about 161 milligrams per day of the mixture.

### EXAMPLE 2

16.0 grams of Saw Palmetto Extract containing between 80.0% and 95.0% of fatty acids and between 0.20% to 0.40% of sterols is dissolved in 100 milliliters of 90% ethanol in a 250 ml beaker. To this solution is added 0.03 grams of alfuzosin and admixture is effected by gentle stirring of the solution. Removal of the excess solvent provides a residue suitable for the treatment of benign prostatic hypertrophy. An alternate regiment is about 160 milligrams four times per day of the mixture.

### EXAMPLE 3

16.0 grams of Saw Palmetto Extract containing between 80.0% and 95.0% of fatty acids and between 0.20% to 0.40% of sterols is dissolved in 100 milliliters of 90% ethanol in a 250 ml beaker. To this solution is added 0.10 grams of bunazosin and admixture is effected by gentle stirring of the solution. Removal of the excess solvent provides a residue suitable for the treatment of benign prostatic hypertrophy. A typical dose for an adult human is about 161 milligrams per day of the mixture.

### EXAMPLE 4

16.0 grams of Saw Palmetto Extract containing between 80.0% and 95.0% of fatty acids and between 0.20% to 0.40% of sterols is dissolved in 100 milliliters of 90% ethanol in a 250 ml beaker. To this solution is added 0.025 grams of bunazosin and admixture is effected by gentle stirring of the solution. Removal of the excess solvent provides a residue suitable for the treatment of benign prostatic hypertrophy. An alternate regiment is about 160 milligrams four times per day of the mixture.

### EXAMPLE 5

16.0 grams of Saw Palmetto Extract containing between 80.0% and 95.0% of fatty acids and between 0.20% to 0.40% of sterols is dissolved in 100 milliliters of 90% ethanol in a 250 ml beaker. To this solution is added 0.10 grams of indoramin and admixture is effected by gentle stirring of the solution. Removal of the excess solvent provides a residue suitable for the treatment of benign prostatic hypertrophy. A typical dose for an adult human is about 161 milligrams per day of the mixture.

### EXAMPLE 6

16.0 grams of Saw Palmetto Extract containing between 80.0% and 95.0% of fatty acids and between 0.20% to 0.40% of sterols is dissolved in 100 milliliters of 90% ethanol in a 250 ml beaker. To this solution is added 0.025 grams of indoramin and admixture is effected by gentle stirring of the solution. Removal of the excess solvent provides a residue suitable for the treatment of benign prostatic hypertrophy. An alternate regiment is about 160 milligrams four times per day of the mixture.

### EXAMPLE 7

B 16.0 grams of Saw Palmetto Extract containing between 80.0% and 95.0% of fatty acids and between 0.20% to 0.40% of sterols is dissolved in 100 milliliters of 90% ethanol in a 250 ml beaker. To this solution is added 0.10 grams of prazosin and admixture is effected by gentle stirring of the solution. Removal of the excess solvent provides a residue suitable for the treatment of benign prostatic hypertrophy. A typical dose for an adult human is about 161 milligrams per day of the mixture.

### EXAMPLE 8

16.0 grams of Saw Palmetto Extract containing between 80.0% and 95.0% of fatty acids and between 0.20% to 0.40% of sterols is dissolved in 100 milliliters of 90% ethanol in a 250 ml beaker. To this solution is added 0.025 grams of prazosin and admixture is effected by gentle stirring of the solution. Removal of the excess solvent provides a residue suitable for the treatment of benign prostatic hypertrophy. An alternate regiment is about 160 milligrams four times per day of the mixture.

### EXAMPLE 9

16.0 grams of Saw Palmetto Extract containing between 80.0% and 95.0% of fatty acids and between 0.20% to 0.40% of sterols is dissolved in 100 milliliters of 90% ethanol in a 250 ml beaker. To this solution is added 0.10 grams of doxazosin and admixture is effected by gentle stirring of the solution. Removal of the excess solvent provides a residue suitable for the treatment of benign prostatic hypertrophy. A typical dose for an adult human is about 161 milligrams per day of the mixture.

### EXAMPLE 10

16.0 grams of Saw Palmetto Extract containing between 80.0% and 95.0% of fatty acids and between 0.20% to 0.40% of sterols is dissolved in 100 milliliters of 90% ethanol in a 250 ml beaker. To this solution is added 0.025 grams of doxazosin and admixture is effected by gentle stirring of the solution. Removal of the excess solvent provides a residue suitable for the treatment of benign prostatic hypertrophy. An alternate regiment is about 160 milligrams four times per day of the mixture.

### EXAMPLE 11

16.0 grams of Saw Palmetto Extract containing between 80.0% and 95.0% of fatty acids and between 0.20% to 0.40% of sterols is dissolved in 100 milliliters of 90% ethanol in a 250 ml beaker. To this solution is added 0.10 grams of terazosin and admixture is effected by gentle stirring of the solution. Removal of the excess solvent provides a residue suitable for the treatment of benign prostatic hypertrophy. A typical dose for an adult human is about 161 milligrams per day of the mixture.

### EXAMPLE 12

16.0 grams of Saw Palmetto Extract containing between 80.0% and 95.0% of fatty acids and between 0.20% to 0.40% of sterols is dissolved in 100 milliliters of 90% ethanol in a 250 ml beaker. To this solution is added 0.025 grams of terazosin and admixture is effected by gentle stirring of the solution. Removal of the excess solvent provides a residue suitable for the treatment of benign prostatic hypertrophy. An alternate regiment is about 160 milligrams four times per day of the mixture.

Consideration must be given to the fact that although this invention has been described and disclosed in relation to certain preferred embodiments, obvious equivalent modifications and alterations thereof will become apparent to one of ordinary skill in this art upon reading and understanding this specification and the claims appended hereto. Accordingly, the presently disclosed invention is intended to cover all such modifications and alterations, and is limited only by the scope of the claims which follow.

I claim:

1. A medicinal composition useful in the treatment of benign prostatic hypertrophy formed from components comprising:
a) a first component comprising the extract of the berries of the Saw Palmetto plant; and
b) a second component comprising an alpha-1 adrenergic blocker.

2. A composition as set forth in claim 1 wherein said extract contains between 0.01% and 2.50% sterols, including every hundredth percentage therebetween.

3. A composition as set forth in claim 1 wherein said first component contains between 5.00% and 98.00% of fatty acids, including every hundredth percentage therebetween.

4. A composition as set forth in claim 1 wherein said alpha-1 adrenergic blocker is selected from the group consisting of: Cardura (doxazosin), Flomax (tamsulosin), and Hytrin (terazosin).

5. A composition as set forth in claim 1 wherein said first component is present in any amount between 0.50% and 99.50% by weight based upon the total weight of the composition, including every hundredth percentage therebetween.

6. A composition as set forth in claim 1 wherein said second component is present in any amount between 99.50% and 0.50% by weight based upon the total weight of the composition, including every hundredth percentage therebetween.

7. A composition as set forth in claim 5 wherein said second component is present in any amount between 99.50% and 0.50% by weight based upon the total weight of the composition, including every hundredth percentage therebetween.

8. A composition according to claim 1 wherein said extract contains between 0.20% and 0.40% sterols, including every hundredth percentage therebetween, and wherein said first component contains between 80.00% and 95.00% of fatty acids, including every hundredth percentage therebetween, and wherein the alpha-1 adrenergic blocker is selected from the group consisting of: Cardura (doxazosin), Flomax (tamsulosin), and Hytrin (terazosin).

9. A composition according to claim 1 wherein said extract contains between 0.20% and 0.40% sterols, including every hundredth percentage therebetween, and wherein said first component contains between 80.00% and 95.00% of fatty acids, including every hundredth percentage therebetween, and wherein the alpha 1 adrenergic blocker is Cardura (doxazosin).

10. A composition according to claim 1 wherein said extract contains between 0.20% and 0.40% sterols, including every hundredth percentage therebetween, and wherein said first component contains between 80.00% and 95.00% of fatty acids, including every hundredth percentage therebetween, and wherein the alpha 1 adrenergic blocker is Flomax (tamsulosin).

11. A composition according to claim 1 wherein said extract contains between 0.20% and 0.40% sterols, including every hundredth percentage therebetween, and wherein said first component contains between 80.00% and 95.00% of fatty acids, including every hundredth percentage therebetween, and wherein the alpha 1 adrenergic blocker is Hytrin (terazosin).

12. A process for treating a person afflicted with benign prostatic hypertrophy comprising the steps of:
a) providing a composition according to claim 1; and
b) introducing a first dose of said composition into a human body.

13. A process according to claim 12 wherein said introducing is carried out by a method selected from the group consisting of: transdermal, subcutaneous, intramuscular, orally, or intravenous.

14. A process according to claim 12 further comprising:
c) waiting at least 20 hours and;
d) introducing a second dose of said composition into a human body.

15. A process according to claim 12 wherein the total mass of the composition introduced into said body from step b) is in the range of between 0.10 grams and 75.00 grams, including every hundredth gram therebetween, per introduction.

16. The process according to claim 12 wherein said composition contains a therapeutically effective therapeutic amount of an alpha-1 adrenergic blocker.

17. The process according to claim 12 wherein said composition contains a therapeutically effective amount of an extract from the berries of the Saw Palmetto plant to improve urine flow characteristics.

18. A process for treating a person afflicted with benign prostatic hypertrophy comprising simultaneous administration to such person a combination which includes:
a) a therapeutically effective amount of the extract of the berries of the Saw Palmetto plant for treating benign prostatic hypertrophy; and
b) a therapeutically effective amount of an alpha-1 adrenergic blocker for treating benign prostatic hypertrophy.

19. A process according to claim 18 wherein the alpha-1 adrenergic blocker is selected from the group consisting of: terazosin, doxazosin, prazosin, bunazosin, indoramin, and alfuzosin.

20. A process according to claim 18 wherein said extract contains between 0.20% and 0.40% sterols, including every hundredth percentage therebetween, and wherein said first component contains between 80.00% and 95.00% of fatty acids, including every hundredth percentage therebetween,
